(19) United States
(12) Patent Application Publication
(10) Pub. No.: US 2006/0241494 A1
(43) Pub. Date: Oct. 26, 2006

(54) Methods for detecting abnormal epithelial tissue

(75) Inventor: Mark Bride, Peoria, AZ (US)

Correspondence Address: JEFFER, MANGELS, BUTLER & MARMARO, LLP, 1900 AVENUE OF THE STARS, 7TH FLOOR, LOS ANGELES, CA, 90067, US

(73) Assignee: Zila Pharmaceuticals, Inc., Phoenix, AZ (US)

(21) Appl. No.: 10/564,800

(22) PCT Filed: Sep. 28, 2004
(86) PCT No.: PCT/US04/31963

§ 371 Date: Jan. 12, 2006

(51) Int. Cl. A61B 6/00

(52) U.S. Cl. 600/476

(57) ABSTRACT

The visibility of abnormal tissue under light having wavelength peaks which selectively identify abnormal tissue is enhanced in the presence of normal ambient light by viewing the tissue through lens which transmit the wavelength peaks but block transmission of other wavelengths.

This invention relates to methods for detecting abnormal epithelial tissue, which may harbor tumor phenotypes.

In another respect the invention pertains to improved methods for conducting real time in vivo examinations of epithelial tissue to detect abnormalities which may be cancerous or which may eventually develop invasive cancer.

### BACKGROUND OF THE INVENTION

Patients who delay in obtaining a cancer consultation for at least two months have significantly higher relative hazards of death than do patients with a shorter delay. Thus, if patients are more regularly subjected to effective cancer screening, the mortality risks of cancer would be reduced. Thus, there was a need for a simple, rapid screening test for detecting abnormal mucosal tissue which may harbor tumor phenotypes, which may indicate the presence of or the eventual development of invasive cancer.

Abnormal epithelial tissue can be visually identified and located in real time in vivo using selective light examinations, which are admirably suited for rapid and inexpensive screening carried out as an adjunct to routine medical and dental examinations. Illustratively, U.S. Pat. Nos. 5,179,938 and 5,329,938, incorporated herein by reference, describe instruments equipped with a chemiluminescent light source which radiates in the visible green, blue and, optionally, red spectrums, with spectral peaks at 450, 550 and 580 nm. Under such illumination, with normal ambient light suppressed, abnormal mucosal tissue appears white. Illustratively, such selective light devices for practicing such in vivo examinations are commercially available under the registered trademark VIZILITE® from Zila Pharmaceuticals, Inc., Phoenix, Ariz., USA.

The selective visualization of abnormal mucosal tissue using such light sources is hindered by normal ambient light (daylight or normal artificial light) falling upon the tissue being examined, such that the standard procedure for conducting such examinations calls for darkening the room in which the examination is conducted. This is not only awkward but also may be impossible when the examination is conducted in rooms with large window areas or when other procedures on other patients are being conducted in the same room served by common conventional lighting.

The primary object of the present invention is to provide a method for conducting such selective light examinations can be carried out without darkening the room in which the examination is conducted. I have now discovered selective light examination methods which can be carried out in the presence of normal ambient light.

### BRIEF DESCRIPTION OF THE INVENTION

Briefly, my invention for screening epithelial tissue for possible abnormal tissue comprises illuminating a gross anatomical area of epithelial tissue with a light of preselected wavelengths, that selectively aids in visualizing abnormal tissue sites on said gross area and viewing the illuminated gross area of tissue through filter lens which transmit light in only in these preselected wavelengths, while substantially blocking transmission of ambient light of wavelengths other than these preselected wavelengths, thus enhancing the selective visualization of any abnormal tissue sites in the presence of normal ambient light.

### DETAILED DESCRIPTION OF THE INVENTION

The following examples are presented to enable those skilled in the art to understand and practice the invention and to identify the presently preferred embodiments thereof. These examples are provided for illustrative purposes and not to indicate the scope of the invention which is defined only by the appended claims.

### Example 1

A routine visual examination of the oral cavity is made, noting the presence of any lesions on the attached gingiva, the buccal mucosa, the floor of the mouth, the hard and soft palate and the dorsal, lateral and ventral tongue. Any lesions noted by this routine examination are recorded.

### Example 2

After completing the routine examination of Example 1, the patient is then instructed to rinse the mouth with a 1% acetic acid solution for up to one minute and then expectorate.

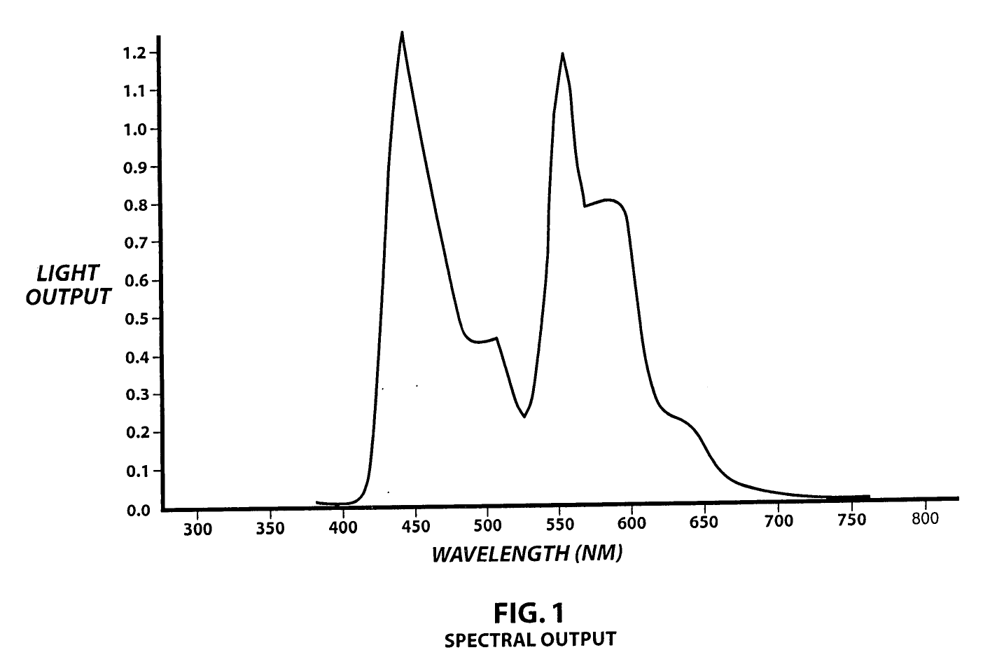

The chemiluminescent light source described in the Lonky patent U.S. Pat. No. 5,329,938, commercially available under the registered trademark VIZILITE®, is activated by bending the flexible outer capsule, breaking the brittle inner vial. The capsule is then shaken and it is inserted into the retractor. The light provided has spectral peaks at about 450 nm, 550 nm and a smaller peak in the red region at about 600 nm, as indicated in FIG. 1. These spectral peaks produce a bluish-white light.

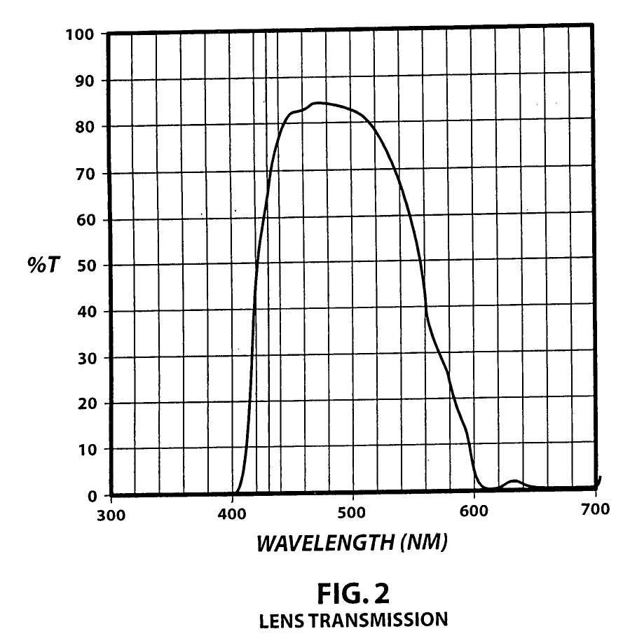

The examining clinician then dons a pair of spectacles provided with lens which only transmit light in the wavelength band of 400-600 nm, as indicated in FIG. 2. These spectacles are shaped to minimize illumination reaching the examiner's eyes from above and from the sides. These spectacles are available commercially under the registered trademark VIZELITE® from Zila Pharmaceuticals, Inc., Phoenix, Ariz.

Without reducing ambient light from normal illumination sources, the visual examination of the oral cavity is then repeated using the illumination provided by the light source, looking for lesions or other suspect tissue sites which appear white, paying special attention to any suspect tissue sites noted in the routine examination of Example 1. Any sites which appear white or bluish-white are noted and recorded.

Further assessment of the noted sites is made, for example by tissue biopsy for standard histology or by molecular analysis, to determine whether the tissue is cancerous or harbors mutations which are in the pathway for eventual development of invasive cancer.

Having described by invention in such terms as to enable those skilled in art to understand and practice it and, having identified the presently preferred embodiments thereof, I CLAIM:

1. The method of screening epithelial tissue for possible abnormal tissue sites, said method comprising:
(a) illuminating a gross anatomical area or epithelial tissue with a light of preselected wavelengths that selectively aids in visualizing abnormal tissue sites on said gross area; and
(b) viewing said gross area through filter lens which transmit light in said preselected wavelengths, while substantially blocking transmission of light of wavelengths other than said preselected wavelengths, to enhance the visualization of any of said abnormal tissue sites in the presence of normal ambient light.

2. A method of detecting abnormal epithelial tissue, comprising:
illuminating an area of epithelial tissue with light having at least one preselected wavelength such that the light is reflected from the area, thereby creating reflected light;
filtering the reflected liht to substantially remove wavelengths other than the at least one preselected wavelength, thereby creating filtered light; and
viewing the filtered light.

3. The method of claim 2, further comprising determining if the filtered light is white.

4. The method of claim 3, wherein if the filtered light is white, the method further comprises performing an assessment of the area, wherein the assessment is one selected from the group consisting of a tissue biopsy, a histological analysis, or a molecular analysis.

5. The method of claim 2, wherein the at least one preselected wavelength is from about 400 nm to about 600 nm.

6. The method of claim 2, wherein the abnormal epithelial tissue includes tumor phenotypes.

7. The method of claim 2, wherein the light further comprises ambient light and the step of filtering substantially removes ambient light.

8. The method of claim 2, wherein the illuminating step comprises directing light emitted from a chemiluminescent light source toward the area of epithelial tissue.

9. The method of claim 2, wherein the at least one preselected wavelength comprises a first wavelength of about 450 nm, a second wavelength of about 550 nm, and a third wavelength of about 600 nm.

10. The method of claim 2, further comprising providing spectacles having a filter, and wherein the step of filtering the reflected light comprises filtering the reflected light with the spectacles.
